(19) United States
(12) Patent Application Publication
(10) Pub. No.: US 2005/0054946 A1
(43) Pub. Date: Mar. 10, 2005

(54) Variations of biopsy jaw and clevis and method of manufacture

(76) Inventor: Jacek Krzyzanowski, Etobicoke (CA)

Correspondence Address: MILLER THOMPSON, LLP, 20 QUEEN STREET WEST, SUITE 2500, TORONTO, ON, M5H 3S1, CA

(21) Appl. No.: 10/655,014

(22) Filed: Sep. 5, 2003

(51) Int. Cl. A61B 10/00

(52) U.S. Cl. 600/564

(57) ABSTRACT

A clevis for a biopsy jaw assembly having a pair of biopsy cups, which includes a central portion having a first side and a second side. At least two flaps are oppositely folded to one another with one flap on the first side and one flap on the second side. A mounting portion may be connected to the central portion. The mounting portion may have a proximal end for mounting on an actuator for positioning the biopsy jaw assembly.

### FIELD OF THE INVENTION

This invention relates in general to endoscopic instruments and more particularly to variations of a biopsy jaw and clevis and method of manufacturing same in a more cost efficient manner to produce a superior product.

### BACKGROUND OF THE INVENTION

There are numerous biopsy jaw devices available that are primarily used in the endoscopy field in the removal of tissues from inside the body. The majority of these devices are complex, extremely small and delicate so that the manufacture of parts and assembly requires precision and is costly and time consuming.

Prior art biopsy jaws have been developed to address some of the aforenoted problems, however these devices and methods to do not disclose a simplified device that includes an improved cutting edge, is inexpensive to manufacture and is efficient to assemble. For example, U.S. Pat. No. 6,514,269 issued on Feb. 4, 2003 to Yamamoto and relates to an endoscopic treatment instrument that has cup portions including inner cutting blades on the rims. The angle of inner cutting blade is 85° or more. Due to such cutting blade, tissue uneasily slides thereon and the cup portions can bite tissue more effectively

Simpson et al. is the owner of U.S. Pat. No. 6,149,607, which issued on Nov. 21, 2000 and U.S. Pat. No. 6,139,508, which issued on Oct. 31, 2000. Both patents relate to a biopsy device for acquiring more than one tissue sample comprises an elongated, flexible actuator shaft, which is slidable within an outer sleeve. A jaw portion is coupled to the actuator shaft and the outer sleeve in such a manner as to open when the shaft is moved in a first direction relative to the sleeve and close when the shaft is moved in a second direction relative to the sleeve.

U.S. Pat. No. 5,707,392 issued Jan. 13, 1998 to Kortenbach and relates to a hermaphroditic stamped forceps jaw for disposable endoscopic biopsy forceps and method of making same. The instrument is made from a flat sheet of stainless steel or other suitable material which is stamped and then progressively formed into a jaw cup having a tang. The jaw may also have two tangs that are substantially parallel to one another where one tang is shorter then the other. Both tangs are provided with central mounting holes for mounting the jaw on an axle pin between the arms of the clevis. The longer of the tangs is provided with a hole for coupling it to the distal end of a control wire. The tangs are located such that when the jaws are mounted in the clevis the short tang of each jaw resides between the two tangs of the other jaw.

Thus a biopsy jaw, clevis and method of manufacturing same which may be manufactured more efficiently, less expensively while providing an efficient cutting edge, with less parts to assemble is desirable.

### SUMMARY OF THE INVENTION

An object of one aspect of the present invention is to provide improved variations of a biopsy jaw and clevis, and a method of manufacturing the same.

In accordance with one aspect of the present invention there is provided a clevis for a biopsy jaw assembly having a pair of biopsy cups, which includes a central portion having a first side and a second side. At least two flaps are oppositely folded to one another with one flap on the first side and one flap on the second side. A mounting portion may be connected to the central portion. The mounting portion may have a proximal end for mounting on an actuator for positioning the biopsy jaw assembly.

Conveniently, the central portion may have either a molded portion or a series of spacers instead of the flaps. Preferably, the pair of jaw cups have distal ends and proximal ends, where the distal ends are chamfered, coined cutting edges, and the proximal ends have at least one arm, with the arm being adapted to receive the clevis.

In accordance with another aspect of the present invention there is provided a biopsy jaw having a jaw cup with a distal end and a proximal end. The distal end may have a chamfered, coined cutting edge, while the proximal end may have at least one arm. The arm may further comprise a flap adapted to receive a clevis.

In accordance with another aspect of the present invention there is provided a method of manufacturing a biopsy jaw comprising of cutting a sheet of metal into a strip, punching a series of guide holes into the strip of metal, and defining in the strip of metal a pattern of the biopsy jaw. A portion of the pattern may be cut out so as to expose an edge of the pattern so that the pattern is still attached to the sheet of metal. The exposed edge may be chamfered to deburr any rough edges. The pattern is then folded and shaped into a jaw cup having a concave-shaped bottom portion and a flat cutting edge. The flat cutting edge may then be coined to provide a hardened, sharp cutting edge. The jaw cup may then be folded and shaped into a final shape. A series of holes may then be punched into the pattern of the biopsy jaw. The pattern may be further shaped to form an arm. The pattern may then be cut off of the strip.

In accordance with a further aspect of the present invention there is provided a method of manufacturing a clevis with flaps comprising of punching a series of guide holes into a sheet of metal, and defining in the sheet of metal a pattern of the clevis with flaps. A portion of the pattern may be cut out so as to expose an edge of the pattern so that the pattern is still attached to the sheet of metal. The exposed edge may be chamfered to deburr any rough edges. A series of holes may then be punched into the pattern of the biopsy jaw. The pattern may be folded and shaped to form the clevis with flaps.

Advantages of the present invention are: less parts, more efficient design, coined cutting edge makes for a harder edge, coined exposed edges deburred early on during manufacture for increased efficiency, progressive stamping method efficient, less overall costs, flap arrangement on clevis itself for increased efficiency when assembling; chamfered edges early on in method of manufacture to improve production with less chance of device catching within the endoscope channel.

### BRIEF DESCRIPTION OF THE DRAWINGS

A detailed description of the preferred embodiments are provided herein below by way of example only and with reference to the following drawings, in which:

FIG. 1 in a perspective view, illustrates a clevis for a biopsy jaw assembly having a pair of biopsy cups in accordance with a preferred embodiment of the present invention.

FIG. 2 in a perspective view, illustrates the clevis with biopsy jaw cups in an open position.

FIG. 2A in a cross-sectional view along the line 2A of FIG. 2, illustrates the chamfered edge of the biopsy jaw cup.

FIG. 3 is an exploded view of a preferred embodiment of FIG. 1.

FIG. 4 is a top plan view of a preferred embodiment of FIG. 1.

FIG. 4A is a side elevational view of a preferred embodiment of FIG. 1.

FIG. 5 is a top plan view of a preferred embodiment of FIG. 2.

FIG. 5A is a side elevational view of a preferred embodiment of FIG. 2.

FIG. 6 is a perspective view of a clevis for a biopsy jaw assembly having a pair of biopsy cups in accordance with a second preferred embodiment of the present invention.

FIG. 7 is an exploded view of the clevis with biopsy jaw cups of FIG. 6.

FIG. 8 is a perspective view of a clevis for a biopsy jaw assembly having a pair of biopsy cups in accordance with a third preferred embodiment of the present invention.

FIG. 9 is an exploded view of the clevis with biopsy jaw cups of FIG. 8.

FIG. 10 is a perspective view of a biopsy jaw cup with flap in accordance with a fourth embodiment of the present invention.

FIG. 10A is a perspective view of the biopsy jaw cup of FIG. 10 and its interaction with a clevis.

FIG. 10B is a perspective view of the biopsy jaw cups and clevis assembled in an open position with a pivot pin mounted from the outside of the biopsy jaw cup.

FIG. 11 is a perspective view of the embodiment shown in FIGS. 10A and 10B where the pivot pin is mounted from within the biopsy jaw cup.

FIG. 11A is a cross-sectional view along the line 11A of FIG. 11 showing the positioning of the pivot pin.

FIGS. 12, 12A and 12B are top plan views of the various biopsy jaw cups.

FIG. 13 is a perspective view of a clevis for a biopsy jaw assembly having a pair of biopsy cups in accordance with a fifth embodiment of the present invention.

FIG. 13A is a cross-sectional view of an alternative arrangement of the clevis.

FIG. 13B is a cross-sectional view of an alternative arrangement of the clevis.

FIG. 14 is a perspective view of a clevis in accordance with a sixth embodiment of the present invention.

FIGS. 15A, 15B, 15C, 15D, and 15E illustrates in perspective views the series of steps during the method of manufacture.

FIG. 16 is a perspective view of the bottom side of the pattern of the biopsy jaw during the method of manufacture in accordance with a preferred embodiment of the invention as shown in FIG. 15B.

In the drawings, preferred embodiments of the invention are illustrated by way of example. It is to be expressly understood that the description and drawings are only for the purpose of illustration and as an aid to understanding, and are not intended as a definition of the limits of the invention.

### DETAILED DESCRIPTION OF THE PREFERRED EMBODIMENT

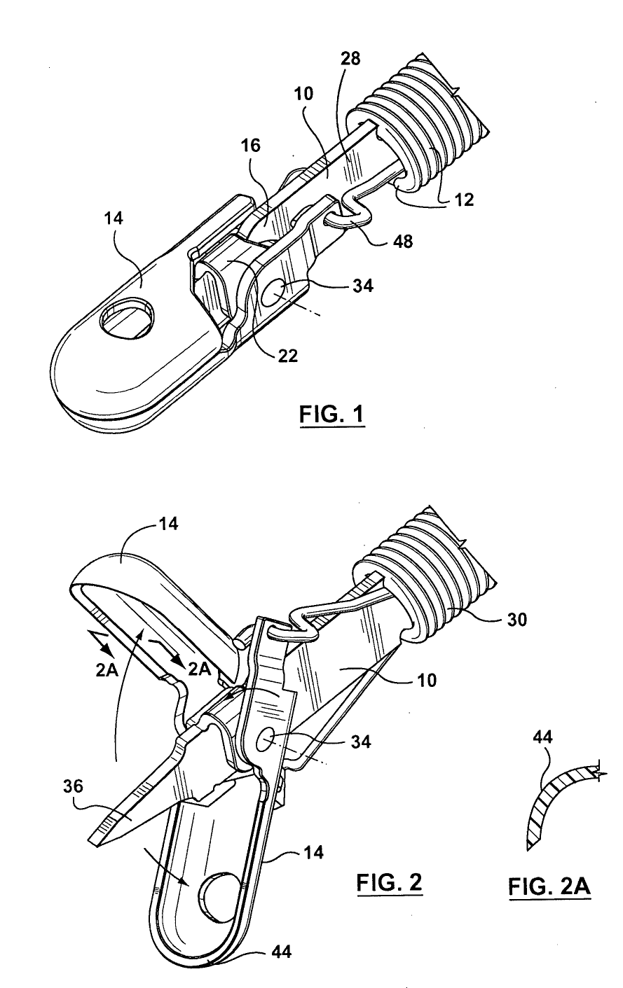

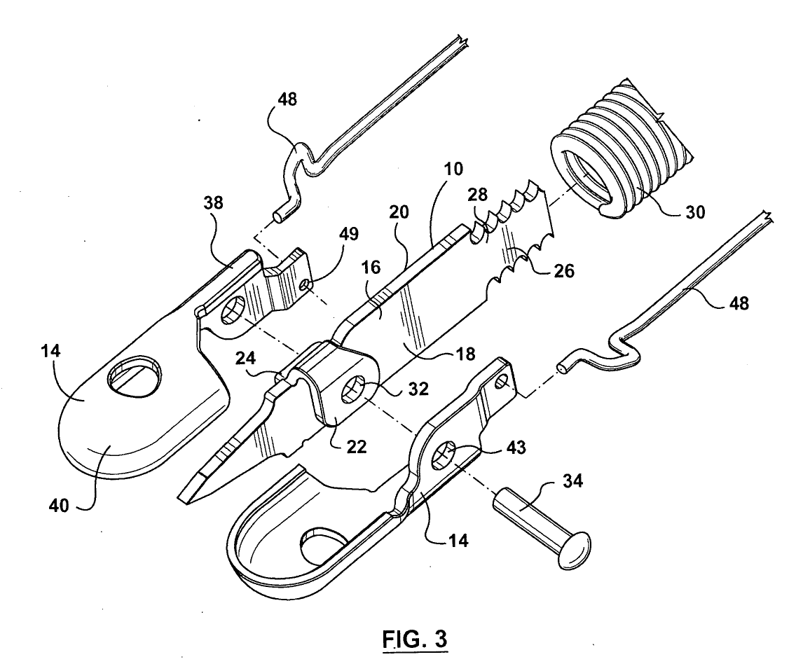

Referring to FIGS. 1, 2, 2A and 3, there is illustrated in perspective, cross-sectional and exploded views, a clevis 10 for a biopsy jaw assembly 12 having a pair of biopsy cups 14 in accordance with a preferred embodiment of the present invention. The clevis 10 includes a central portion 16 having a first side 18 and a second side 20. At least two flaps 22 and 24 respectively, are oppositely folded to one another with one flap 22 on the first side 18 and the second flap 24 on the second side 20. A mounting portion 26 may be connected to the central portion 16. The mounting portion 26 may have a proximal end 28 for mounting on an actuator 30 for positioning the biopsy jaw assembly 12.

The flaps 22 and 24 respectively allow for a more efficient design for the biopsy jaw assembly 12. At least one pivot hole 32 may be located through the flaps 22 and 24 respectively and the central portion 16 of the cleivs 10. The pivot hole 32 can receive a pivot pin 34 so as to mount the pair biopsy cups 14 to the clevis 10. The clevis 10 may have a distal end 36 that is pointed or the distal end 36 may be blunt.

The biopsy cups 14 may be further defined as having proximal ends 38 and distal ends 40. The proximal ends 38 may have at least one arm 42 that is adapted to receive the clevis 10. More specifically the arm 42 may have a pivot hole 43 so as to receive the pivot pin 34. The distal ends 40 may have chamfered or beveled coined cutting edges 44 as seen in FIG. 2A. The cutting edges 44 may or may not be serrated.

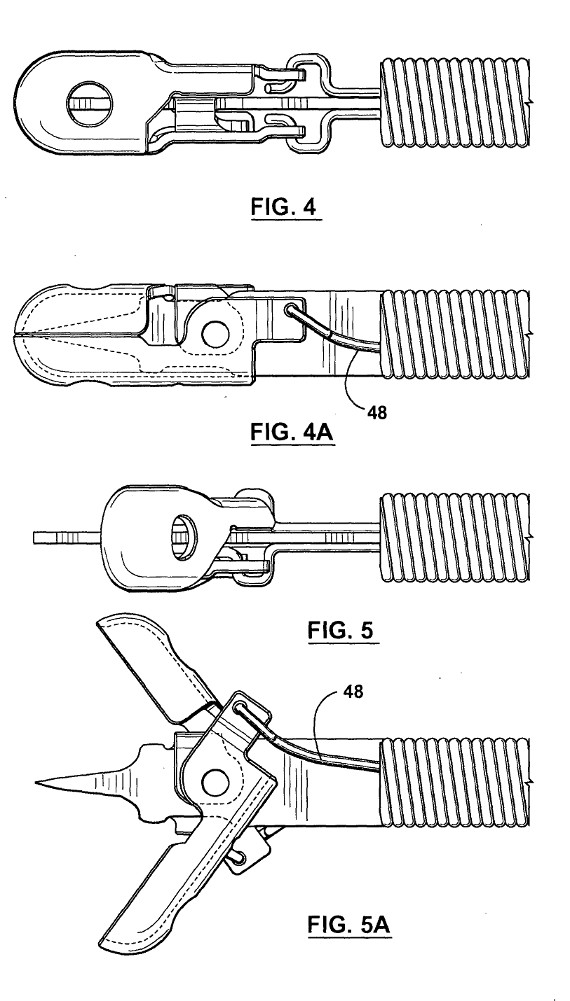

Referring to FIGS. 4, 4A, 5, and 5A, the biopsy jaw assembly 12 may be actuated by a pair of actuating wires 48 attached through at least one actuating hole 49 located on the arm 42 in such away to allow the biopsy jaw assembly 12 to open and close. Specifically the actuating hole 49 is located above the plane of the cutting edge 44 to allow for the opening and closing action to take place. The actuating wires 48 move the biopsy jaw cups from a closed position to an open position.

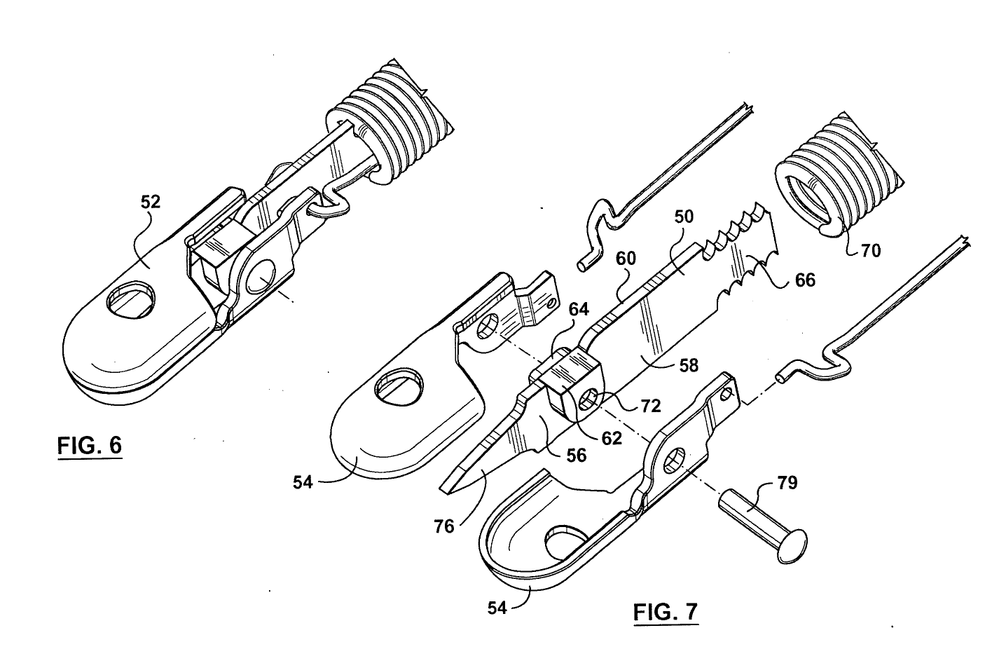

Referring to FIGS. 6 and 7 there is illustrated in perspective and exploded views, a clevis 50 for a biopsy jaw assembly 52 having a pair of biopsy cups 54 in accordance with a second preferred embodiment of the present invention. The clevis 50 includes a central portion 56 having a first side 58 and a second side 60. At least two molded portions 62 and 64 respectively, are positioned so that one molded portion 62 is on the first side 58 and a second molded portion 64 on the second side 60.

A mounting portion 66 may be connected to the central portion 56. The mounting portion 66 may have a proximal end 68 for mounting on an actuator 70 for positioning the biopsy jaw assembly 52.

At least one pivot hole 72 may be located through the molded portions 62 and 64 respectively and the central portion 56 of the cleivs 50. The pivot hole 72 can receive a pivot pin 74 so as to mount the pair biopsy cups 52 to the clevis 50. The clevis 50 may have a distal end 76 that is pointed or the distal end 76 may be blunt. The biopsy cups 54 may be configured as described above for the first embodiment.

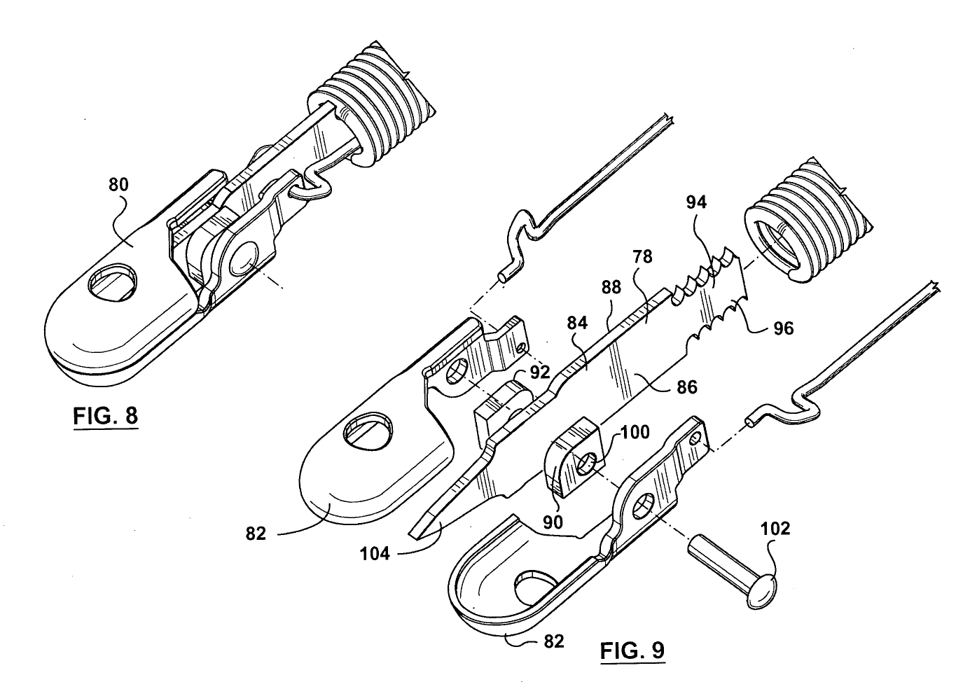

Referring to FIGS. 8 and 9, there is illustrated in perspective and exploded views, a clevis 78 for a biopsy jaw assembly 80 having a pair of biopsy cups 82 in accordance with a third preferred embodiment of the present invention. The clevis 78 includes a central portion 84 having a first side 86 and a second side 88. At least two spacers 90 and 92 respectively, are positioned so that one spacer 90 is positioned on the first side 86 and a second spacer 92 is positioned on the second side 88.

A mounting portion 94 may be connected to the central portion 84. The mounting portion 94 may have a proximal end 96 for mounting on an actuator 98 for positioning the biopsy jaw assembly 80.

At least one pivot hole 100 may be located through the spacers 90 and 92 respectively and the central portion 84 of the cleivs 78. The pivot hole 100 can receive a pivot pin 102 so as to mount the pair biopsy cups 82 to the clevis 78. The clevis 78 may have a distal end 104 that is pointed or the distal end 104 may be blunt. The biopsy cups 82 may be configured as described above for the first embodiment.

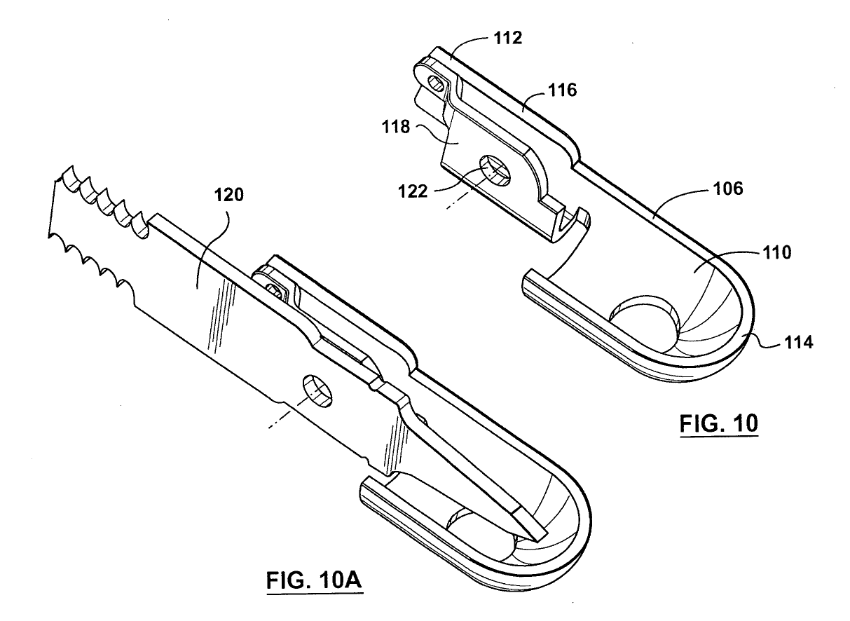

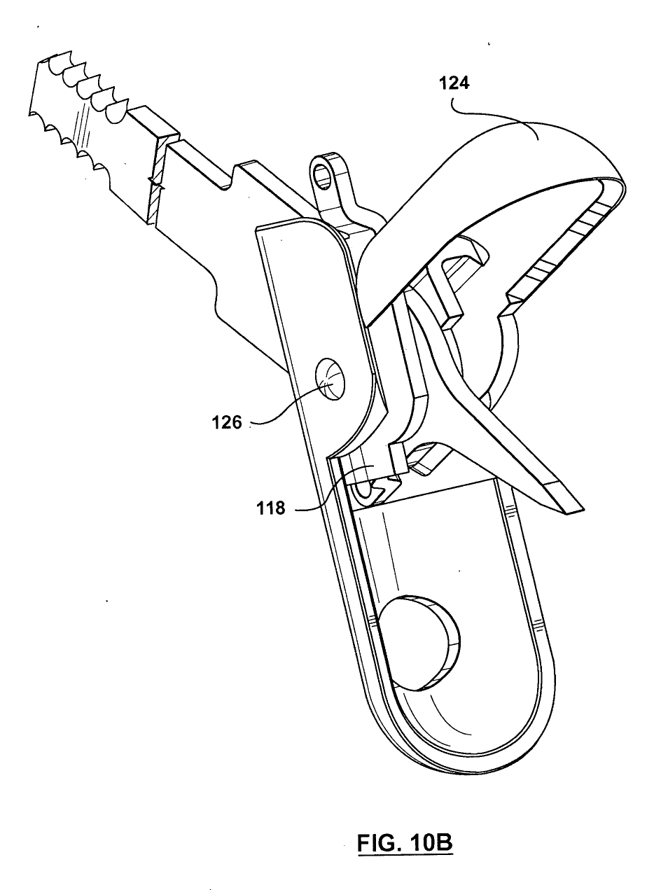

Referring to FIGS. 10 and 10A there is illustrated in perspective views, a biopsy jaw cup 106 in accordance with a fourth embodiment of the present invention. The biopsy cup 106 may have a jaw cup 108 with a distal end 110 and a proximal end 112 and exposed edges 113 that have been chamfered. The chamfering of the exposed edges 113 eliminates any rough edges that may damage the channel of the endoscope. The distal end 110 may have a chamfered, coined cutting edge 114, while the proximal end 112 may have at least one arm 116. The arm 116 may further comprise a flap 118 that is adapted to receive a clevis 120. The flap 118 may further include a pivot hole 122 for mounting the biopsy cup 106 to the clevis 120. The arm 116 may also have a pivot hole 122.

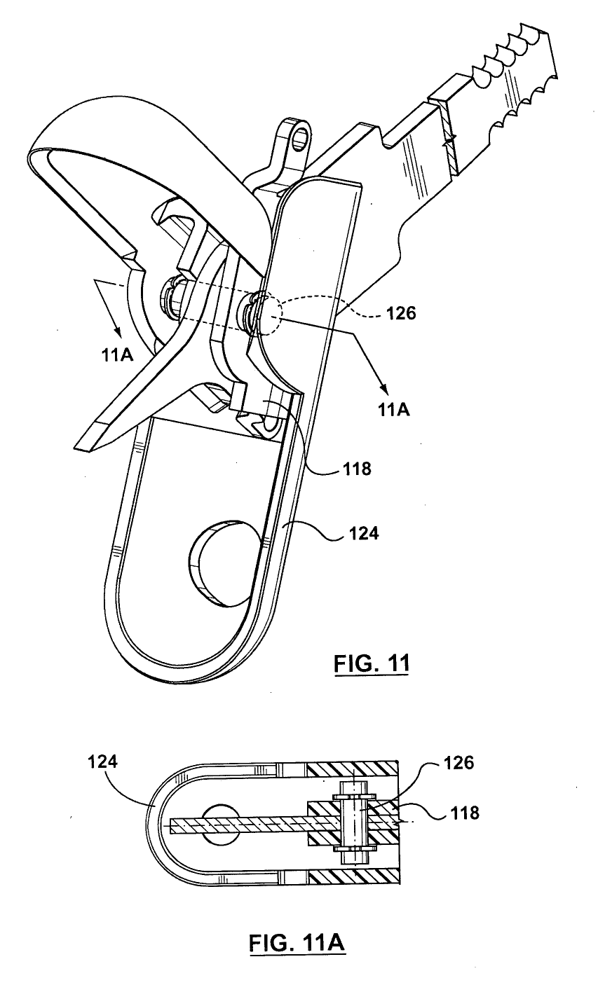

Referring to FIGS. 10B, 11, and 11A there is illustrated in perspective and cross-sectional views, the biopsy cup 106 assembled into a biopsy jaw assembly 124. FIGS. 11 and 11A illustrate the biopsy cup 116 with a pivot pin 126 mounted through the clevis 120 and the flap 118 only. FIG. 10B illustrates the biopsy cup 116 with the pivot pin 126 mounted through the flap 118 and the arm 116 of the biopsy cup 116.

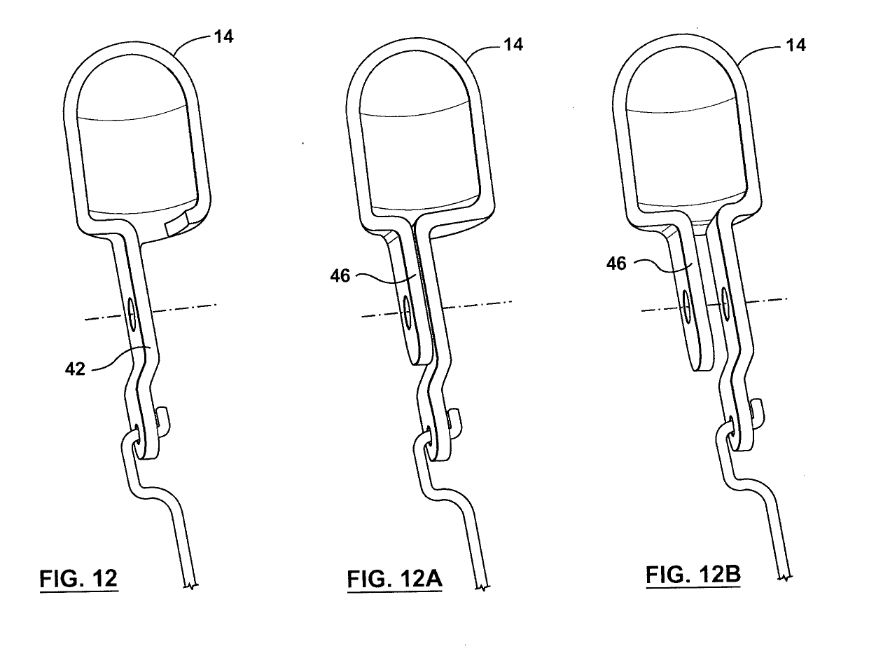

Referring to FIGS. 12, 12A and 12B, the biopsy jaw cups 14 may be configured to have one arm 42 or a second arm 46. The configuration of the arms 42 or 44 depends on the various embodiments.

The biopsy cups 14 may be mounted to the clevis 10 in a variety of ways depending on the desired configuration of the biopsy jaw assembly 12. Referring to FIG. 1, this embodiment illustrates the biopsy cups 14 mounted around the flaps 22 and 24 respectively of the clevis 10. This configuration is also illustrated in FIGS. 6 and 8 for the second and third embodiments.

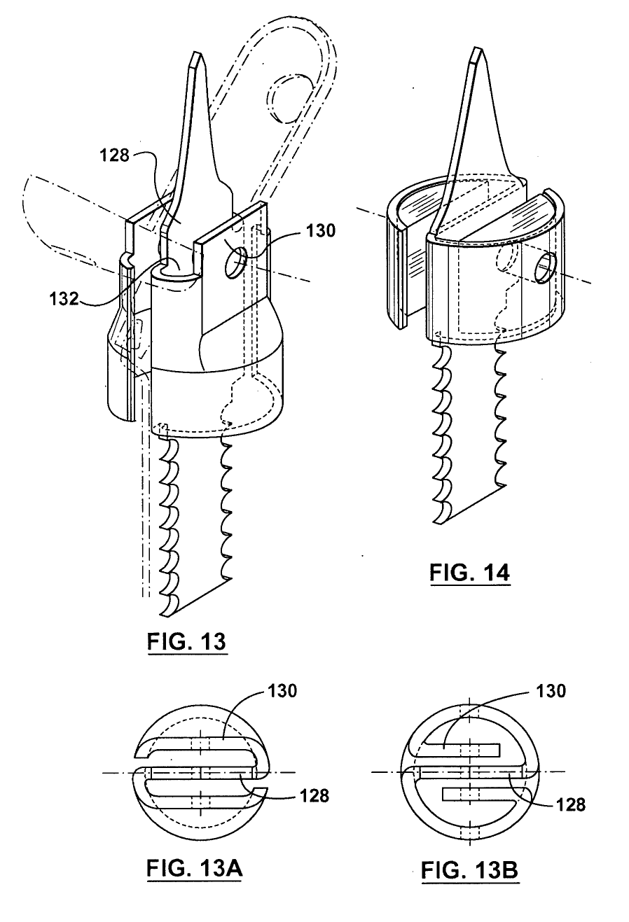

FIGS. 13, 13A, 13B and 14 illustrate in perspective and cross-sectional views, the clevis 128 configured so as to allow the biopsy cups 14 to be mounted in between the flaps 130 and the central portion 132 of the clevis 128. Various configurations of the clevis 128 are possible to as to allow the clevis 128 to act as an insert into the actuator 30 and to be attached to the actuator 30 through various methods.

The clevis 10, 50, 78 and 128 and more specifically the mounting portion 26, 66 and 94 may be attached to the actuator 30, 70 and 98 through a variety of methods such as: screwing the mounting portion 26, 66, and 94 into the actuator; friction fitting the mounting portion 26, 66 and 94 into the actuator 30, 70 and 98; or crimping the mounting portion 26, 66 and 94 into the actuator 30, 70 and 98 or any similar method.

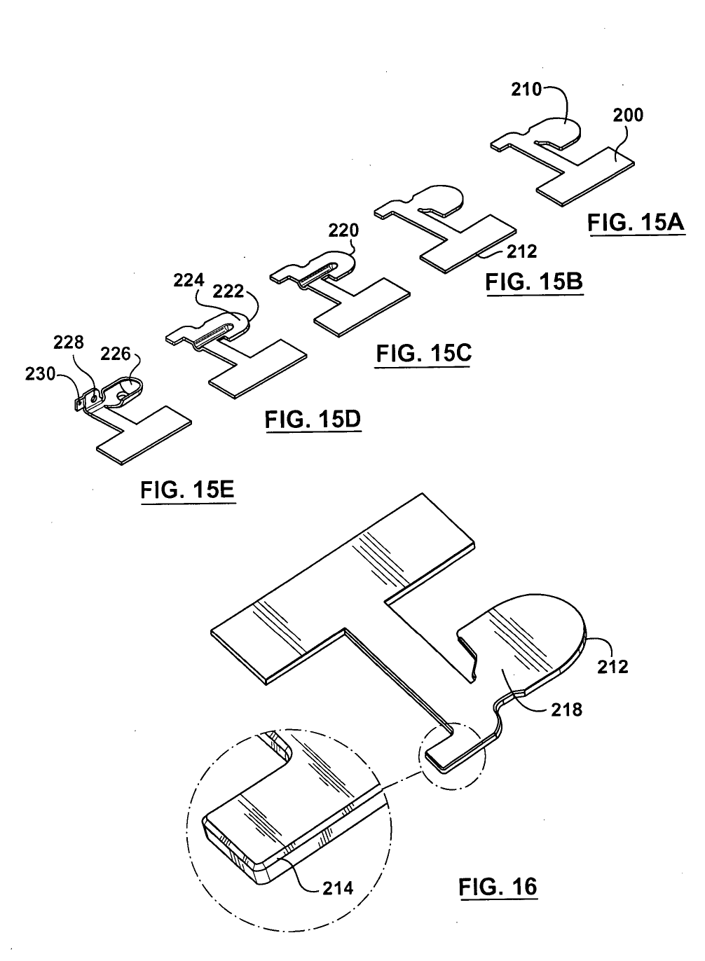

In accordance with another aspect of the present invention there is provided a method of manufacturing a biopsy jaw described above through a progressive die stamping method. Referring to FIGS. 15A, 15B, 15C, 15D, and 15E there is illustrated in perspective views the series of steps during the method of manufacture. A sheet of metal is initially cut into a desired strip. The strip is then punched with a series of guide holes. The guide holes locate or guide the strip of metal through the different stages of the progressive die stamping manufacturing process.

Referring to FIG. 15A, a pattern 200 of the biopsy jaw is defined in the strip of metal. A portion of the pattern 210 is cut out exposing an edge 212 of the pattern 200 but leaving a portion of the pattern still attached to the strip of metal. Referring to FIG. 15B, the exposed edge 212 may then be chamfered to debur any rough edges and/or exposed sharp corners resulting in a smooth exposed edge 214. FIG. 16 shows the chamfering of the edges 212 which occurs on the bottom side 218 of the pattern 200. The chamfering happens as a result of the stamping process.

Referring to FIGS. 15C, 15D and 15E, the flat cutting edge 220 is coined and chamfered to provide a hardened, sharp cutting edge 222 at the distal end 224 of the jaw cup 226. The sharp cutting edge 222 may be serrated as well. The pattern 200 is then folded and shaped into a jaw cup 226. More specifically the bottom of the part of the jaw cup 226 is pre-formed into a concave shape. The remainder of the jaw cup 226 is then folded and shaped. More specifically the jaw cup 226 is hit from the sharp cutting edge side with a shaped punch so as to ensure that the sharp cutting edge 222 is positioned in one plane and to ensure the perpendicularity of the cutting edge plane to the arm(s) of the jaw cup 226. It may also be possible to form the jaw cup 226 first and then coin and sharpen the cutting edge 222 after forming the jaw cup 226.

Referring to FIG. 15E a series of holes 228 and fenstrations may then be punched into the pattern of the jaw cup 226 creating the pivot holes and the like. The pattern 200 may then be further shaped to form an arm 230 of the biopsy jaw cup 226. The jaw cup 226 is then cut from the strip of metal.

The chamfering of the exposed edges 212 of the pattern 200 results in a biopsy cup that is completely smooth thereby reducing the number of sharp edges that may protrude from the biopsy cup and possibly damage the channel of the endoscope through which the biopsy cup is passed. Through the chamfering process, this potential problem with sharp edges catching on the channel of the endoscope is removed.

The chamfering and hardening of the cutting edge of the jaw cup improves the cutting done by the biopsy jaw assembly so better quality specimen can be obtained.

The progressive die stamping method described above may also be used to manufacture a clevis with flaps as described above. The addition of the flaps on the clevis results in a more efficient design during the stamping process. The method may comprise of taking a sheet or strip of metal is initially punched with a series of guide holes. The guide holes locate or guide the strip of metal through the different stages of the progressive die stamping manufacturing process. A pattern of the clevis with the flaps is defined in the sheet of metal. A portion of the pattern is cut out exposing an edge of the pattern but leaving a portion of the pattern still attached to the strip of metal. The exposed edge may then be chamfered to debur any rough edges resulting in a smooth exposed edge. The pattern may then be folded and shaped into a clevis with flaps in a variety of configurations as illustrated in FIGS. 13, 13A, 13B and 14.

A hole or series of holes may then be punched into the pattern of the cleis with flaps creating the pivot holes and the like. The pattern may then be further shaped to form the desired configuration of the clevis with flaps. The chamfering of the exposed edges of the pattern results in a clevis that is completely smooth thereby reducing the number of sharp edges that may protrude from the clevis and possibly damage the channel of the endoscope through which the clevis is passed.

Other variations and modifications of the invention are possible. All such modifications or variations are believed to be within the sphere and scope of the invention as defined by the claims appended hereto.

1. A clevis for a biopsy jaw assembly having a pair of biopsy cups, comprising:
(a) a central portion having a first side and a second side and at least two flaps oppositely folded to one another with one flap on said first side and one flap on said second side; and
(b) a mounting portion connected to said central portion, said mounting portion having a proximal end for mounting on an actuator for positioning said biopsy jaw assembly.

2. A clevis as claimed in claim 1 wherein said central portion further comprises at least one pivot hole through said flaps and said central portion for mounting said biopsy cups.

3. A clevis as claimed in claim 2 wherein said central portion has a pointed distal end.

4. A clevis as claimed in claim 3 wherein said biopsy cups further comprise two jaw cups having distal ends and proximal ends, said distal ends having chamfered, coined cutting edges, and said proximal ends having at least one arm, said arm adapted to receive said clevis.

5. A clevis as claimed in claim 4 wherein said biopsy cups are mounted around said flaps on said first and second sides of said central portion of said clevis.

6. A clevis as claimed in claim 4 wherein said biopsy cups are mounted in between said flaps and said first and second sides of said central portion of said clevis.

7. A clevis as claimed in claim 4 wherein said proximal end of said mounting portion is configured so as to be mounted to said actuator by screwing-in method, friction-fit method or crimping method.

8. A clevis for a biopsy jaw assembly having a pair of biopsy cups, comprising:
(a) a central portion having a first side and a second side and having a molded portion attached on both said first and second sides of said central portion; and
(b) a mounting portion connected to said central portion, said mounting portion having a proximal end for mounting on an actuator for positioning said biopsy jaw assembly.

9. A clevis as claimed in claim 8 wherein said central portion further comprises at least one pivot hole through said molded portion and said central portion for mounting said biopsy cups.

10. A clevis as claimed in claim 9 wherein said central portion has a pointed distal end.

11. A clevis as claimed in claim 10 wherein said biopsy cups further comprise two jaw cups having distal ends and proximal ends, said distal ends having chamfered, coined cutting edges, and said proximal ends having at least one arm, said arm adapted to receive said clevis.

12. A clevis as claimed in claim 11 wherein said proximal end of said mounting portion is configured so as to be mounted to said actuator by screwing-in method, friction-fit method or crimping method.

13. A clevis for a biopsy jaw assembly having a pair of biopsy cups, comprising:
(a) a central portion having a first side and a second side and having a spacer on both said first and second sides of said central portion; and
(b) a mounting portion connected to said central portion, said mounting portion having a proximal end for mounting on an actuator for positioning said biopsy jaw assembly.

14. A clevis as claimed in claim 13 wherein said central portion further comprises at least one pivot hole through said spacers and said central portion for mounting said biopsy cups.

15. A clevis as claimed in claim 14 wherein said central portion has a pointed distal end.

16. A clevis as claimed in claim 15 wherein said biopsy cups further comprise two jaw cups having distal ends and proximal ends, said distal ends having a chamfered, coined cutting edge, and said proximal ends having at least one arm, said arm adapted to receive said clevis.

17. A clevis as claimed in claim 16 wherein said proximal end of said mounting portion is configured so as to be mounted to said actuator by screwing-in method, friction-fit method or crimping method.

18. A biopsy jaw comprising:
(a) a jaw cup having a distal end, a proximal end, and exposed chamfered edges;
(b) said distal end having a chamfered, coined cutting edge; and
(c) said proximal end having at least one arm, said arm further comprising a flap adapted to receive a clevis.

19. A biopsy jaw as claimed in claim 18 wherein said flap further comprises a pivot hole for mounting said biopsy jaw to said clevis.

20. A biopsy jaw as claimed in claim 19 wherein said flap and said arm further comprise a pivot hole for mounting said biopsy jaw to said clevis.

21. A method of progressive die stamping manufacturing a biopsy jaw comprising:
(a) Cutting a sheet of metal into a strip;
(b) Punching a series of guide holes into said strip of metal;
(c) Defining in said sheet of metal a pattern of said biopsy jaw;
(d) Cutting out a portion of said pattern and exposing an edge of said pattern wherein said pattern is attached to said strip of metal;
(e) Chamfering said exposed edge;
(f) Coining said flat cutting edge to provide a hardened, sharp cutting edge;
(g) Folding and shaping said pattern into a jaw cup having a concave-shaped bottom portion and a flat cutting edge;
(h) Folding and shaping said jaw cup into a final shape;
(i) Punching a series of holes into said pattern of said biopsy jaw;
(j) Shaping said pattern of said biopsy jaw to form an arm; and
(k) Cutting said jaw cup off of said strip.

22. A method of progressive die stamping manufacturing a clevis with flaps comprising:
(a) Punching a series of guide holes into a sheet of metal;
(b) Defining in said sheet of metal a pattern of said clevis with flaps;
(c) Cutting out a portion of said pattern and exposing an edge of said pattern wherein said pattern is attached to said sheet of metal;
(d) Chamfering said exposed edge;
(e) Punching a series of holes into said pattern of said clevis with flaps; and,
(f) Folding and shaping said pattern to form said clevis with flaps.
